(19) United States
(12) Patent Application Publication
(10) Pub. No.: US 2020/0155070 A1
(43) Pub. Date: May 21, 2020

(54) EXERGAMING FOR THE PREVENTION OF VENOUS THROMBOEMBOLISM (VTE)

(71) Applicant: Baylor College of Medicine, Houston, TX (US)

(72) Inventors: Jayer Chung, Houston, TX (US); Bijan Najafi, Houston, TX (US); Hadi Rahemi, Houston, TX (US)

(21) Appl. No.: 16/616,238

(22) PCT Filed: May 21, 2018
(86) PCT No.: PCT/US18/33629

§ 371 Date: Nov. 22, 2019

Related U.S. Application Data
(60) Provisional application No. 62/509,484, filed on May 22, 2017.

(51) Int. Cl. A61B 5/00; A61B 5/11; A61H 1/02; A61B 5/026

(52) U.S. Cl. CPC A61B 5/6829; A61B 5/1118; A61B 5/6802; A61B 5/7455; A61B 5/742; A61H 1/02; A61H 2209/00; A61B 5/7475; A61B 5/026; A61B 5/486; A61H 2201/5007; A61B 2505/09; A61B 5/4848

(57) ABSTRACT

A game-based platform for exercise procedures for prevention of venous thromboembolism (VTE) may have a wearable sensor and human-machine interface technology configured to monitor and encourage VTE prevention exercises. The wearable sensor may attach to a patient's foot to monitor movement of the ankle joint, such as pronation, supination, dorsiflexion, or plantar flexion. The patient may be instructed to move their foot by operating their ankle to move a cursor around on a screen to reach a target. Targets may be randomly presented to keep the patient moving their foot for a specified duration of time.

### CROSS-REFERENCE TO RELATED PATENT APPLICATIONS

This application claims the benefit of priority of U.S. Provisional Patent Application No. 62/509,484 to Jayer Chung et al. filed May 22, 2017 and entitled “Exergaming for the Prevention of Venous Thromboembolism (VTE),” which is hereby incorporated by reference.

### GOVERNMENT LICENSE RIGHTS

This invention was made with government support under Grant 1R21CA190933-01A1 awarded by the National Institute of Health (NIH). The government has certain rights in the invention.

### FIELD OF THE DISCLOSURE

The instant disclosure relates to medical diagnostics and intervention. More specifically, certain portions of this disclosure relate to a computerized platform for evaluating venous thromboembolism and/or deep vein thrombosis.

### BACKGROUND

Conventional techniques for preventing venous thromboembolism (VTE) include passive compression stockings, sequential compression devices, and/or anticoagulant medications. However, these conventional measures are uncomfortable, and require formal healthcare settings to administer properly. Thus, compliance has been poor, with deep venous thrombosis continuing to plague inpatient admissions. Some studies approximate in-hospital VTE rates range between 1.0-3.0% of hospital admissions. VTE remains the most common preventable cause of in-hospital death. Moreover, these measures do not address VTE that occurs in outpatients, which may be as much as 30-50% of VTE that occur after major intra-abdominal surgery. Exercise has also been recommended to prevent DVT, but adherence to exercise is difficult to enforce, particularly for outpatients. In addition, weight bearing exercise is often impractical (e.g. for bed bound patients, patients out of hospital, or for those who are taking long distance flight). Most importantly, accurate execution of exercise tasks without real-time feedback to the user is currently impossible, which reduces the effectiveness of exercise tasks to prevent VTE. Furthermore, without management of timing, range of motion, and frequency of exercise tasks, muscle fatigue may occur, which in turn may reduce the effectiveness of exercise to improve venous flow. While supervised exercise could address some of these limitations, it is expensive and impractical. Each of these conventional preventative treatments for VTE are also not suitable for routine usage in busy clinics and/or outside of clinic including nursing homes and other long term settings where staff is not available to oversee each patient's exercise routines.

### SUMMARY

A computerized platform for venous thromboembolism (VTE) prevention may include a wearable sensor and a human-machine interface technology configured to implement preventative measures for patients, guide, and encourage the patients to accurately execute and complete each preventative exercise. The wearable sensor may attach to the patient, such as on a leg or foot, or may be injected inside of the user. Any one of those sensors allows measuring movement of a joint of interest, in some embodiments in real-time. The movement of sensor may be visualized in a human-machine interface to allow guiding and encouraging execution of specific lower extremity motor tasks designed to prevent VTE. The human-machine interface may be a mobile device, personal computer, smartwatch, tablet, TV, electronic tattoo devices, injectable devices, or another computing device communicating with the wearable sensor. The human-machine interface may provide feedback to a user using either visual or non-visual source such as vibration and audio feedback. The feedback allows controlling speed, duration, frequency, range of motion, and number of consecutive lower extremities motor tasks to increase the benefit of the exercise task from the point of view of VTE treatment, while reducing the duration of needed exercise tasks to avoid muscle fatigue.

One example procedure implemented through the human-machine interface may involve requesting a patient to complete tasks by navigating a cursor between circles on a computer screen using the wearable sensor. The number of consecutive VTE preventative measures may include one or more tasks involving moving a cursor between targets, such as circles, other shapes, or images, appearing on a computer screen, in which the tasks may be completed by moving the patient's limb with the wearable sensor attached. For example, a patient may rotate their ankle left and right along one axis and up and down along a perpendicular axis. The wearable sensor may report data, such as a measured rotation of an ankle joint (e.g., dorsiflexion-extension) to confirm completion of the tasks. The interface may guide the user to flex and extend ankle joint with a pre-defined range of motion to navigate a cursor from one target (home target) to another target in a pre-defined time interval, from which its virtual distance from the home target is defined based the needed ankle range of motion to be effective for the purpose of preventing VTE. The timing, range of motion, and number of consecutive tasks could be personalized based on the user's demography (e.g., age, gender, BMI, muscle calf circumference, etc.) to avoid fatigue while maximizing benefit. Similarly, a personalized pause (e.g., time break) could be added between two consecutive exercise tasks to ensure relaxation of calf muscle. This may be done by managing the timing, in which a new target appears on the human-machine interface. Different visual and/or audio and/or vibratory feedbacks may be provided to a user after execution of each single motor task (e.g., completing a single ankle flexion or extension task) to guide the user whether he/she executed the task within a good time frame with suitable velocity and range of motion. In addition, other feedback mechanisms could be provided at the end of each task execution to inform the user about the percentage of a pre-set goal, he/she has achieved at that point based on the performance of previously executed motor tasks. Furthermore, an optional feedback may be provided to the user via text message, calendar setting, etc. to remind the user about next exercise session. The timing of the reminder may be adjusted based on the execution of movement tasks in a previous session, demography information, clinician recommendation, and/or the patient's risk for VTE.

Furthermore, an optional mechanical or electrical stimulation could be activated by the platform in response to output of the sensors. The stimulation may magnify muscle activation, leg compression, and/or lower extremity blood flow. An optional sensor may also be used to monitor efficacy of the platform to increase blood flow and personalized the exercise to maximize the benefit. For example, a Doppler sensor may be used to measure changes in velocity and volume blood flow or return flow to a lower extremity and guide the user to perform an exercise that will maximize the blood flow or flow return. Alternatively, the platform could measure muscle contraction in a body segment of interest to determine the efficacy of the exercise or personalize the exercise task, including its range of motion, speed, acceleration, and timing. In this example, and certain other embodiments, feedback from one or more sensors is used to adjust the exercise task, such as by personalizing the exercise to improve the likelihood of obtaining a desired result.

Data collected from the wearable sensor and other information regarding the completed tasks may be stored by the human-machine interface and processed or transferred to a server for processing. Healthcare providers may access the stored data or access summaries of the stored data to monitor adherence to the VTE prevention therapy, and alert both the patient and the healthcare providers as to how well the patient is adhering to the required therapy. The patient may also be provided information to self-modify their own risk of VTE as an inpatient or an outpatient. Self-modification may include modifying the frequency of exercise and/or the appropriateness of the specific range of motion exercises performed. For example, if the patient misses a circle, or does not flex the calf sufficiently to activate the circle, the game can provide a “score” that informs the patient of the motions required to achieve a better score the next time. The data may be accessed through a computer interface, such as a web page or a mobile application on mobile devices, any of which may allow for healthcare providers to monitor the patient's adherence in the outpatient setting. The healthcare provider may update the required therapy for the patient by modifying the tasks to be presented to the patient. This allows healthcare providers to precisely modify the patient's outpatient care to prevent VTE. The patient may also access results, modify the exercise routines, and/or receive updates from their healthcare providers through the computer interface or mobile application.

Venous flow may be monitored for one or more or all of the femoral veins during the patient's completion of the tasks with pre-defined kinematic patterns. The calf venous pump is the strongest in the human body, and the rationale behind the computer-monitored tasks is to improve venous flow, thereby preventing VTE. However, not every patient has a calf-pump that is strong enough to produce sufficient flow into the femoral vein. The presented tasks allow healthcare providers to readily identify which subjects can produce sufficient venous flow with exercise alone, versus those subjects that may benefit from further adjuncts. Venous flow may be monitored with the same wearable sensor configured to monitor limb movement. Venous flow may alternatively or additionally be measured with an additional wearable senor. In other embodiments, venous flow may be measured by other equipment under operation by a trained healthcare provider and the venous return flow measurements correlated with the presented tasks. Venous return flow may be monitored through parameters such as blood flow volume and peak and mean blood flow velocity.

The patient's exercises may be personalized by measuring venous flow after exercise in a clinic and having a healthcare provider access the computer interface or mobile application. For example, not everyone has the same calf pump. In the clinic, a healthcare provider can measure the venous flow with a duplex ultrasound in response to the game-based exercise. The measurements allow identification of patients that are potentially at higher risk of deep venous thrombosis and alter the game parameters to make the patient perform exercises that improve the venous flow for that patient better than the default game settings. Such customization of the game-based exercise procedure can also be performed automatically in response to information acquired from the wearable sensor or by prompting the patient with questions about how they feel after the exercise.

The stored data may also be used to identify patients at risk of deep vein thrombosis (DVT). DVT is the manifestation of venous thromboembolism (VTE). Together with pulmonary embolism (PE) are the leading cause for preventable hospital deaths and the cause of as many as 100,000 premature deaths in the United States. Up to 60% of the patients develop VTE after surgical discharge, and the majority of these patients are not provided with preventative measures because the patient's risk is not identified and/or executing the preventative measures is too difficult for an outpatient.

According to one embodiment, a method may include presenting a series of requested motions to be performed by a patient; receiving motion data from a sensor recording the patient performing the series of requested motions; analyzing the received motion data to determine a compliance of the patient with the requested motions; and/or reporting completion of the requested motions. The requested motions may be part of an exercise program for the prevention of VTE. The requested motions may be randomly presented to the user, with the number of requested motions, the range of requested motions, and/or the timing for completing a requested motion prescribed by a healthcare provider.

The method may be programmed as a computer program product for execution by a computing device to carry out certain steps of the method. The method may be carried out by a system comprising a wearable sensor comprising at least one motion sensor, wherein the wearable sensor is configured to record motion data from the at least one motion sensor and to transmit the motion data, and a computing device configured to receive the motion data transmitted by the wearable sensor and configured to perform certain steps of the method. The wearable sensor communicate with the computing device through a wired connection or through wireless communications circuitry for wireless communication via Bluetooth or another wireless communications protocol. Additional sensors may be used to monitor venous flow in connection with the requested motions.

As used herein the term “patient” refers to any person capable of completing a venous thromboembolism (VTE) prevention exercise procedure according to any embodiment of the invention disclosed herein.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings.

FIG. 1 is an illustration of a patient with a wearable sensor for interacting with a human-machine interface for administering a venous thromboembolism (VTE) prevention exercise procedure according to one embodiment of the disclosure.

FIG. 2 is an illustration of a graphical user interface (GUI) for a human-machine interface for administering a venous thromboembolism (VTE) prevention exercise procedure according to one embodiment of the disclosure.

FIG. 3 is a flow chart illustrating an example method for a human-machine interface to administer a venous thromboembolism (VTE) prevention exercise procedure according to one embodiment of the disclosure.

FIG. 4A is a graph of flow volume for patients at various times after administration of a venous thromboembolism (VTE) prevention exercise procedure according to one embodiment of the disclosure.

FIG. 4B is a graph of mean velocity for patients at various times after administration of a venous thromboembolism (VTE) prevention exercise procedure according to one embodiment of the disclosure.

FIG. 4C is a graph of peak velocity for patients at various times after administration of a venous thromboembolism (VTE) prevention exercise procedure according to one embodiment of the disclosure.

FIG. 4D is a graph of vein area for patients at various times after administration of a venous thromboembolism (VTE) prevention exercise procedure according to one embodiment of the disclosure.

FIG. 5A is a graph of normalized flow volume for male and female patients at various times after administration of a venous thromboembolism (VTE) prevention exercise procedure according to one embodiment of the disclosure.

FIG. 5B is a graph of normalized mean velocity for male and female patients at various times after administration of a venous thromboembolism (VTE) prevention exercise procedure according to one embodiment of the disclosure.

FIG. 5C is a graph of normalized peak velocity for male and female patients at various times after administration of a venous thromboembolism (VTE) prevention exercise procedure according to one embodiment of the disclosure.

FIG. 5D is a graph of normalized vein area for male and female patients at various times after administration of a venous thromboembolism (VTE) prevention exercise procedure according to one embodiment of the disclosure.

FIG. 6 is a graph identifying groupings of patients with respect to blood flow parameters based on percentage change in post-exercise peak velocity after administration of a venous thromboembolism (VTE) prevention exercise procedure according to one embodiment of the disclosure.

FIG. 7 is a graph illustrating ankle velocity differences in patients of two groups measured during administration of a venous thromboembolism (VTE) prevention exercise procedure according to one embodiment of the disclosure

### DETAILED DESCRIPTION

Game-based kinematic exercises may be implemented as a venous thromboembolism (VTE) prevention exercise procedure. The exercises may be performed by a patient wearing a wearable sensor. The computerized exercise procedures managed by a human-machine interface allow the patient and healthcare providers to personalize VTE prevention. Furthermore, the technology allows VTE prevention to be performed in the outpatient setting. Thus, a patient may be more likely to complete VTE prevention exercise procedures as they may be able complete the procedure at a facility close to their home, or even from the comfort of their own home. The game-based nature of the exercises may also motivate patients to perform ankle exercises. The patient's ankle movements in response to the game-based prompts may be recorded with real-time biofeedback. The VTE prevention may be used for managing deep vein thrombosis (DVT). The game-based platform not only allows for a long-lasting improvement in venous return blood flow, but also allows for a longer, more convenient, period of exercise without fatiguing the patients.

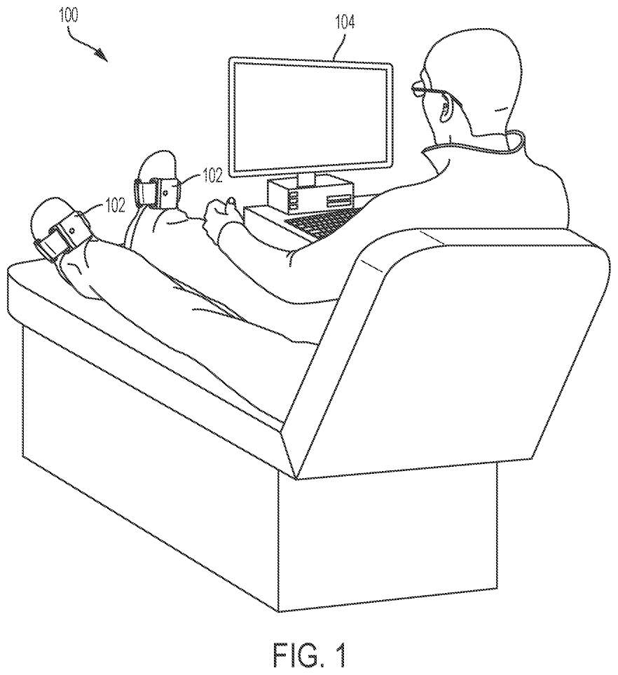

FIG. 1 is an illustration of a patient with a wearable sensor interacting with a human-machine interface for administering range of motion exercises according to one embodiment of the disclosure. A system 100 may include a wearable sensor 102, which may include one or more of an accelerometer, a gyroscope, and a magnetometer sensor. The sensors may be configured to measure inertial signals (e.g. acceleration, angular velocity, angle) for assessing a range of motion movement of patient movement during presented tasks. In some embodiments, the sensor 102 may be combined with other sensor units such as heart rate monitoring, respiration monitoring, or venous return flow monitoring. The sensing components of the wearable sensor 102 may be configured for estimation of angles and position of the wearable sensor 102 during VTE prevention exercises, such as to track a patient's foot movements. The wearable sensor 102 may also include a processor, memory, and communications circuitry (such as a wireless transceiver/receiver) for processing data from the sensing components and transmitting the sensor data to a human-machine interface. In some embodiments, the data could be processed in another location such as cloud, tablet, cell phone, or computers. In some embodiments, the transmitted sensor data may be processed data, such that the sensor data includes angles and positions or another representation of the data received from the sensing components. In one embodiment, the wearable sensor 102 may be a wireless inertial sensor, which can include a 3D accelerometer, a 3D gyroscope, and/or a 3D magnetometer on a dorsum of each of the patient's feet.

The sensor 102 may track patient appendage motion in three dimensions while the patient moves his foot (e.g., ankle joint rotations) to navigate a computer cursor on the human-machine interface 104 to execute exercise tasks. The sensor 102 may be used by the patient as an input interface for human-machine interface 104 to control or manipulate images and/or information displayed on a display connected to the human-machine interface 104. The patient's progress in completing exercise tasks may be monitored and reflected in real time by the human-machine interface, thus motivating the patient to complete the tasks. The human-machine interface 104 and/or sensor 102 can provide visual, haptic, and/or audio feedback to the user as the user executes exercise tasks. Patients do not need to be supervised during the exercise session because the tasks and monitoring are computerized. Patients may remain seated or recumbent during the presented tasks. Performance may be tracked automatically and data stored, processed, and/or uploaded to a server. Although only one wearable sensor 102 is shown in FIG. 1, sensor data may be collected from multiple locations on a patient, such as by the addition of additional wearable sensors to the patient.

The wearable sensor 102 may be configured in a housing with attachment devices (such as string, Velcro, elastic straps, wraps, etc.) to attach to the patient. In one embodiment, the wearable sensor 102 may be housed in compression hose. During an exercise procedure, the wearable sensor 102 may be attached to the patient's shin or on top of the patient's foot. In some embodiments, the sensor could be implemented inside of body, such as by injecting a small sensor under the skin. The movement of the wearable sensor 102 may be transmitted to a human-machine interface 104 installed on a personal computer or other processing device. In some embodiments, portions of the human-machine interface may be integrated with the wearable sensor 102. For example, the wearable sensor 102 may cast information to a nearby display to present tasks to the patient, collect sensor data during administration of the test, analyze the sensor data to determine the patient's compliance, and display a result of the test on the cast display or a display screen or other indicator integrated with the wearable sensor 102. In some embodiments the human-machine interface 104 may be a computer laptop, desktop, tablet, cell phone, TV, eye-glasses, virtual reality goggles, or any other means of visualization. In some embodiments, the human-machine interface 104 may be connected to the wearable sensor 102 via a wired connection, integrated into the wearable sensor, or connected to the wearable sensor 102 via wireless communications circuitry. In some embodiments, the feedback can be non-visual signals such as audio or vibratory feedbacks or combination of visual and non-visual feedbacks.

During an exercise program, the human-machine interface 104 may present a series of requested motions to be performed by a patient. The requested motions may include random rotation amounts for the patient to turn their ankle in one or more directions. By moving the ankle with attached wearable sensor 102, the patient navigates a cursor on the screen to targets appearing on the same screen. The human-machine interface 104 may evaluate the amount of time the patient takes to navigate the cursor to the target and make determinations regarding the patient based on the time for each target in the administered test. Some intermediate results may be displayed during the administration of the test. If a patient is completing tasks too slowly, the patient may be encouraged to try to move their ankle faster. The targets can be any shape or image and may be customized to keep a patient's attention on the test by selecting objects interesting to the patient. In some embodiments, the targets may be personalized for a patient based on the patient's interests or preferences. In some embodiments, a score may be displayed and may increase based on the accuracy and/or speed with which the patient completes tests.

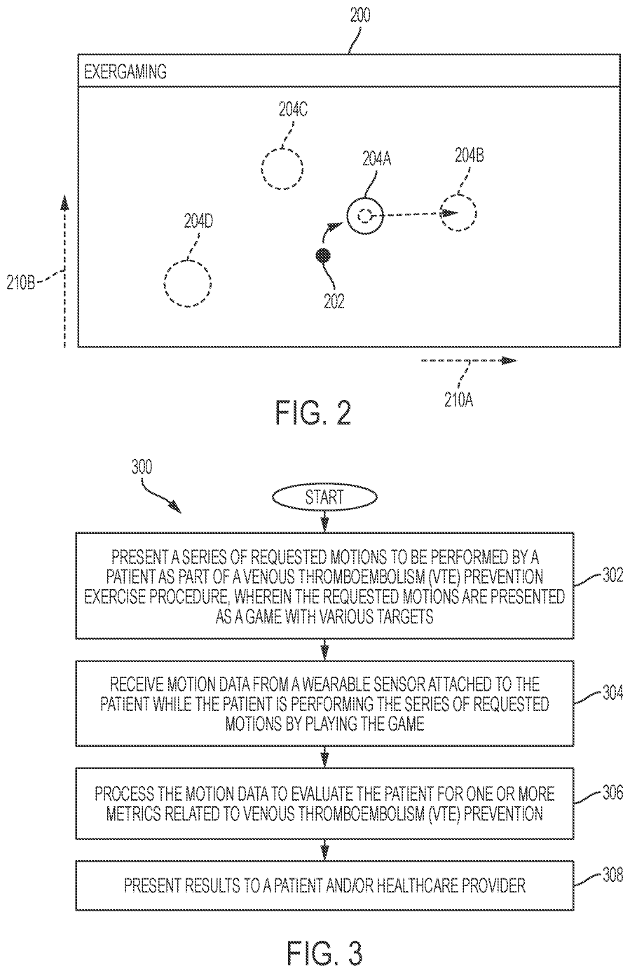

FIG. 2 is an illustration of a graphical user interface (GUI) for a human-machine interface for administering a venous thromboembolism (VTE) prevention exercise procedure according to one embodiment of the disclosure. A screen shot 200 illustrates a graphical user interface (GUI) for the human-machine interface 104 for engaging with a patient and encouraging completion of a venous thromboembolism (VTE) prevention exercise procedure. A cursor 202 may be moved around the screen by tilting the patient's foot. For example, pronation or supination of the ankle may move the cursor 202 along a first direction 210A and dorsiflexion or plantar flexion of the ankle may move the cursor 202 along a second direction 210B. Thus, the user has control to move the cursor 202 anywhere along the screen. A target 204A may appear to instruct the patient to move their foot to move the cursor 202 to an approximate center of or inside of the target 204A. After the patient is successful in moving the cursor 202 to target 204A, an animation may play on the screen to indicate the user reached the target 204A. For example, the target 204A may change colors or pulsate. The target 204A may then disappear and the target 204B appear. The patient must then move the cursor 202 to target 204B, after which the process repeats for targets 204C and 204D. The GUI of screen shot 200 may be presented as a game, such as by replacing the targets 204A-D with fruit and instructing the patient to move the cursor 202 to slice through each piece of fruit. In some embodiments, the wearable sensor may provide other feedback, such as haptic feedback, when a target is reached. For example, the system may, in addition to presenting the cursor 202 slicing through a piece of fruit or other visual effects, cause the sensor to vibrate when the target is reached, or to vibrate with increasing intensity as the target is approached. The game presented in screen shot 200 may be personalized or customized for a patient, such as by allowing a patient to select the type of fruit that appears in the game. In some embodiments, the game presented in screen shot 200 may sync the presentation of targets for user movement to music. In another embodiment, the game presented may direct the patient to guide the cursor 202 along a pathway or through a maze or to avoid obstacles with the cursor 202.

An example method is shown in FIG. 3 for guiding a patient through a VTE prevention exercise. FIG. 3 is a flow chart illustrating an example method for a human-machine interface to administer a venous thromboembolism (VTE) prevention exercise procedure according to one embodiment of the disclosure. A method 300 may begin with sensor calibration, establishing a communication link with the wearable sensor 102, or other initialization steps (not shown). The method 300 may then proceed with administering one or more tests to the patient. At block 302, a human-machine interface may present a series of requested motions to be performed by a patient as part of a VTE prevention exercise. For example, targets may be presented to a patient wearing the wearable sensor 102 and the patient asked to turn their ankle to move a cursor to reach the target. The targets may be presented as part of a game. Next, at block 304, motion data may be received from the wearable sensor attached to the patient while the patient is performing the series of requested motions presented at block 302. During the exercise procedure, the received motion data may be processed to move the cursor on the screen in accordance with the patient's movements. The motion data may also be stored in memory for analysis, such as at block 306. The analysis of block 306 may include, for example, analyzing the received motion data to determine accurate execution of the series of requested motions, providing feedback to the patient to guide the patient to accurately execute the series of requested motions, and/or analyzing the received motion data to determine compliance with venous thromboembolism (VTE) preventive measures. Any of the analysis performed at block 306 may be provided to the patient and/or healthcare providers for review. The analysis at block 306 may be performed after the exercise session or in real-time. When analyzed in real-time, feedback may be provide to the patient as visual, audio, textual, or vibratory feedback. For example, feedback may be provided to the patient as a game score that is incremented in real time. Alternatively or additionally, feedback may be provided to the patient as a final score when a session of testing is complete. The steps 302 and 304 may be repeated to test the patient multiple times using the same test or multiple times using different tests. Some intermediate results and/or further instruction may be presented to the patient during steps 302 and 304.

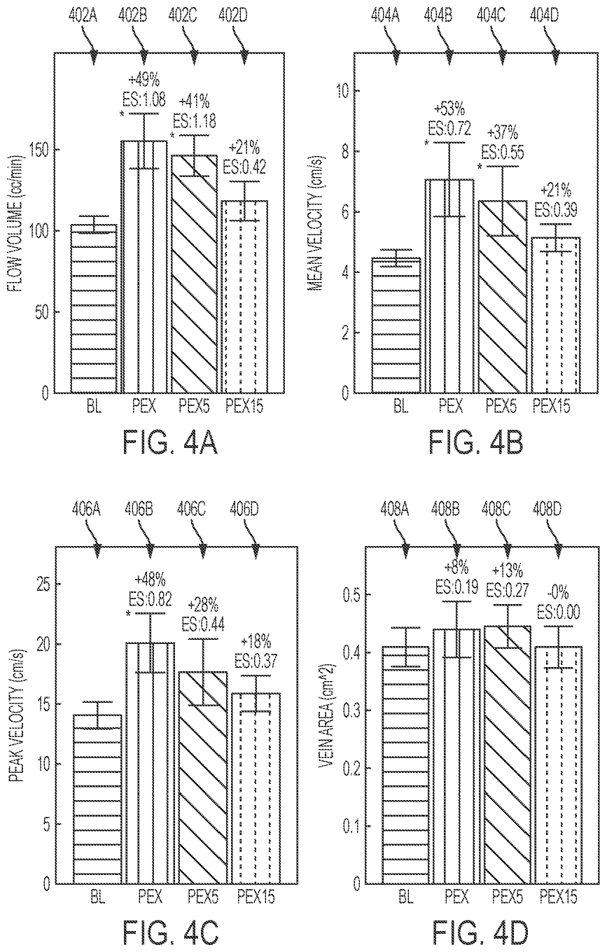

After the patient performs the exercises administered during steps 302 and 304, the human-machine interface may process the motion data to evaluate the patient for one or more metrics. At block 306, the motion data received during the course of the test may be analyzed to determine one or more metrics related to venous thromboembolism (VTE) prevention. The metrics may include, for example, any of the metrics described herein with reference to FIGS. 4A-D, 5A-D, 6, or 7. As another example, motion data may be processed to determine a compliance score for the patient, such as how much of the exercise procedure was correctly completed and/or how diligent a patient is in completing an assigned schedule of exercise procedures. Analysis at block 306 may include analyzing raw sensor data or analyzing summaries of the sensor data recorded during the exercises. For example, times to navigate to each target circle may be stored during the test, and analysis at block 306 may include, for example, averaging the time required for the patient to navigate to each target circle or calculating a total time required to complete the presented tasks.

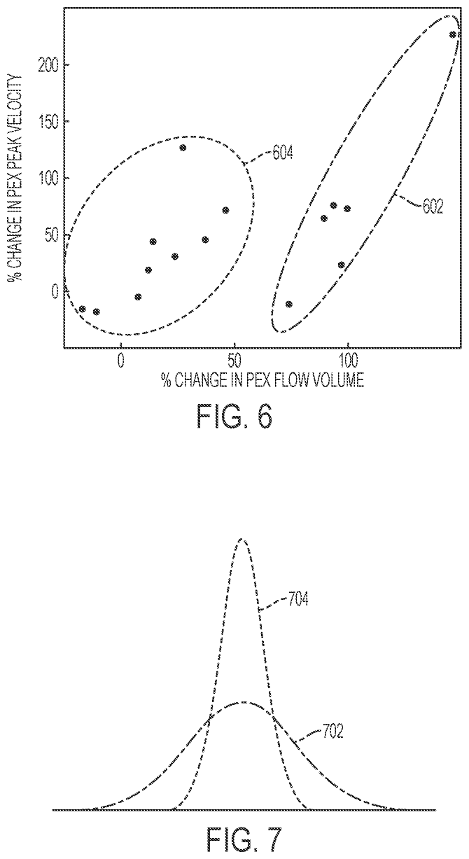

Data gathered during the exercise procedure may be used to evaluate a patient's risk for deep vein thrombosis (DVT). For example, ankle movements may be analyzed, such as by comparing the movements with known movements of low responders and high responders as described herein with reference to FIG. 6 and FIG. 7. In some embodiments, a sensor may measure venous blood flow during the exercise program. The human-machine interface may receive venous flow data corresponding to the received motion data. The received venous flow data may be analyzed to determine a risk for deep vein thrombosis (DVT). For example, one or more of a flow volume, mean velocity, peak velocity, and vein area may be measured before, during, and/or after the exercise procedure. The values may be analyzed to determine whether a patient is a low responder or a high responder to the exercise procedure, such as described herein with reference to FIGS. 4A-D and 5A-D. A low responder may be at higher risk for deep vein thrombosis (DVT) because the exercise procedures are not effective at improving the patient's venous blood flow.

Next, at block 308, the results of the test may be presented to the patient and/or healthcare provider. The results may be presented on a screen after the game is complete. The results may alternatively or additionally be uploaded to a server for access by a patient and/or a healthcare provider. Additional sensor data, such as from venous blood flow measurements, may be measured at times throughout the exercise. Venous blood flow measurements may be processed to determine a patient's risk factor for DVT. In some embodiments, the presenting of results at block 308 may include providing a notification to notify the patient of a next exercise session according to performance and duration of a previous exercise session, clinician recommendation, patient's risk, or patient demographical information. The notification may be, for example, a text message or a calendar invitation, and the notification may be repeated to remind the patient of the next exercise session.

One embodiment of a VTE prevention platform was administered to test subjects. During the test, the triceps surea muscles of each patient were scanned using a B-mode ultrasonography in sagittal plane while the patient was standing. The view showing the maximum depth of each muscle was captured to allow for comparison of muscle thickness and its possible effect on blood flow properties. Wireless EMG electrodes were placed on belly of lateral (LG) and medial (MG) gastrocnemius, lateral (L-Sol), and medial (M-Sol) sides of the soleus, as well as the belly of tibialis anterior (TA).

The patients were asked to lay on the bed in a supine position, and the patients' backs were raised approximately 30 degrees from flat state. The game-based exercises were performed on the right ankle of the patients. Ten minutes of a rest and relaxation period was given to each patient to bring the blood flow to normal conditions. Before starting the game-based exercises, three repeated baseline measures of the blood flow volume peak flow velocity, mean flow velocity, and vein cross sectional area in the right femoral vein was measured by a Doppler ultrasonography technician.

The EMG measurements and game-based exercise were initiated immediately after the baseline (BL) measurements during which each patient guided a cursor on the screen to the center of circular targets by flexion and extension of their right ankle. A total of 200 targets were presented to the patients during the testing, although more or less targets may be implemented in different embodiments of the invention. Patients were instructed to move as fast as possible between the targets. The EMG recording was stopped after the exercise procedure was completed. The exercise duration was recorded for each patient. Ankle angular velocity was also recorded using the wearable sensor and a linear velocity calculated for the foot at each instance using anthropometric tables.

The blood flow parameters in the right femoral vein was repeatedly measured three time at three instances after the game-based exercise. These measurements occurred immediately post exercise (PEX), five minutes after exercise (PEX5), and fifteen minutes after exercise (PEX15). The blood flow results can be expressed as a mean plus or minus a standard deviation (SD). A Mann-Whitney U test is used to compare blood flow parameters after exercise (PEX, PEX5, and PEX15) with baseline (BL) measurements. A percentage of difference between PEX, PEX5, and PEX15 with BL along with Cohen's d effect size (ES) are calculated by processing the wearable sensor data and/or venous blood flow measurements.

For EMG analysis the average intensity of bursts, median frequency of power spectrum, and mean of peak frequency in each burst over the whole period of exercising was calculated. Additionally, the same parameters were calculated for the first half and second half of the exercise to monitor changes that may indicate fatiguing during the exercise. The exercise procedure can be administered for between three to four minutes, although other times may be implemented.

FIG. 4A, FIG. 4B, FIG. 4C, and FIG. 4D summarize blood flow volume, peak velocity, mean velocity, and vein area values at baseline and post exercise measurements. In FIG. 4A, blood flow volume baseline is shown in bar 402A with after exercise flow shown in bar 402B, flow five minutes after exercise in bar 402C, and flow fifteen minutes after exercise in bar 402D. In FIG. 4B, mean velocity baseline is shown in bar 404A with after exercise velocity shown in bar 404B, mean velocity five minutes after exercise in bar 404C, and mean velocity fifteen minutes after exercise in bar 404D. In FIG. 4C, peak velocity baseline is shown in bar 406A with after exercise peak velocity shown in bar 406B, peak velocity five minutes after exercise in bar 406C and fifteen minutes after exercise in bar 406D. In FIG. 4D, vein area baseline is shown in bar 408A with after exercise vein area shown in bar 408B, vein area five minutes after exercise in bar 408C, and vein area fifteen minutes after exercise shown in bar 408D.

Referring to FIG. 4A, blood flow volume showed significant increase during exercise in bar 402B compared to a measured baseline in bar 402A. The increase in blood flow parameters remained significant at PEX5 measurements only for volume flow (P=0.000) shown in bar 402C and mean velocity (P=0.036) shown in bar 404C. Vein area did have a significant response to the exercise at any measured instance. PEX15 measurements, shown in bars 402D, 404D, 406D, and 408D, none of the parameters had a significant increase compared to the baselines shown in bars 402A, 404A, 406A, and 408A. Blood flow volume (P=0.001), peak velocity (P=0.013) and mean velocity (P=0.014) showed significant increase at PEX in bars 402B, 404B, and 406C. These three parameters showed moderate to large effects for all post-exercise measurements.

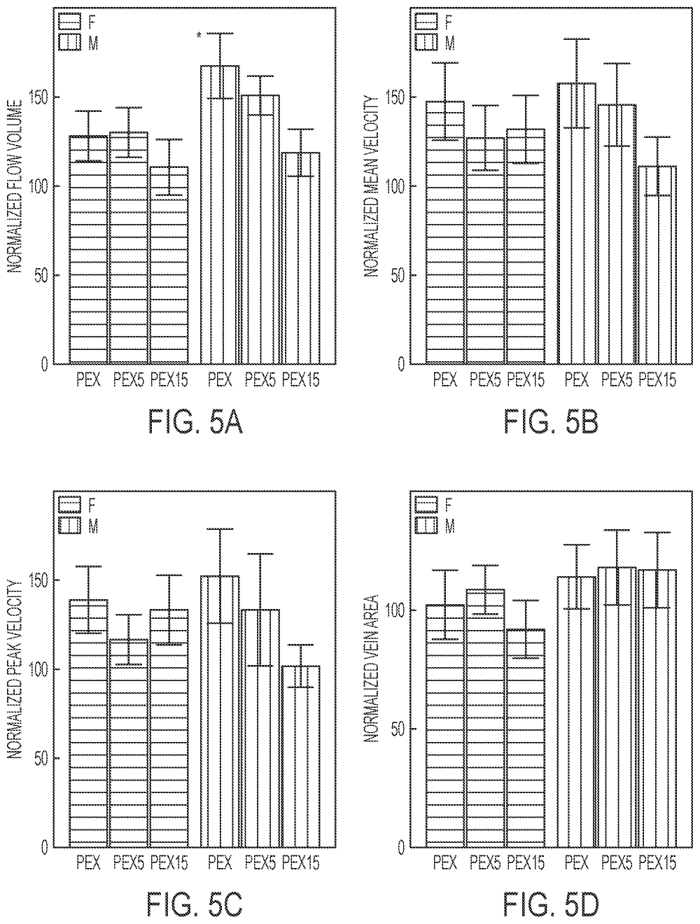

The testing of men and women showed no significant difference between men and women in the blood flow volume (P=0.440), peak velocity (P=0.540), mean velocity (P=0.610), and vein area (P=0.568). All these parameters had a higher normalized value for men at PEX measurement including significant difference in normalized flow volume P=0.042). The values were also higher for men in almost all normalized blood flow parameters at PEX5 and PEX15. A comparison of results for men and women after exercise (PEX), five minutes after exercise (PEX5), and fifteen minutes after exercise (PEX15) is shown in FIG. 5A, FIG. 5B, FIG. 5C, and FIG. 5D for normalized flow volume, normalized mean velocity, normalized peak velocity, and normalized vein area, respectively.

The exercise procedures implemented in embodiments of a venous thromboembolism (VTE) prevention exercise procedure do not fatigue the patient. EMG intensity or the median frequency of power spectrum did not change between measurements in the first half and in the second half of the exercise procedure. None of the measured and calculated EMG and exercise parameters showed high or significant correlation with percent change in blood flow volume.

Patients may be grouped based on their response to the exercise procedure. FIG. 6 shows the percent change in flow peak velocity on a y-axis versus percent change in flow volume on a x-axis. The percent change in after-exercise (PEX) flow volume can be used to define two groups 602 and 604. The group 602 identifies high responder (HR) patients with a relatively high percentage increase in blood flow volume. The group 604 identifies low responder (LR) patients with a relatively low percentage increase in blood flow volume. The generalized linear model showed significant effect from gender (P=0.006) and ethnicity (P=0.004). Of the tested patients, 86% of female participants belong to the LR group and 38% of men belong to the LR group. Despite similar (P=0.516) mean plantar flexion velocity between HR and LR groups, peak plantar flexion velocity of the foot was 16% lower (P=0.046) for the HR group. The median frequency of EMG power spectrum for MG (the largest of triceps surae) was also 55% lower for the HR group. A patient may be categorized as a low responder or a high responder based on one or more of the patient's mean flexion velocity, peak plantar flexion velocity, median frequency of EMG power spectrum for MG or other muscles, percentage change in after-exercise (PEX) flow volume, and/or percentage change in after-exercise (PEX) peak volume. Low responder (LR) patients may be identified through the exercise procedure and assign additional or alternative VTE preventative measures.

Game-based exercise increased the blood flow volume and peak and mean velocity after the exercise. The amount of immediate increase in blood flow parameters was similar to or higher than in conventional VTE prevention techniques. Data collected during the exercise indicates the patients were not physically fatigued during the game-based exercise, which is confirmed by testimonies from the patients that they were not psychologically fatigued. By reducing fatigue, the game-based exercise procedure allows patients to complete the exercises over longer periods because the patients are motivationally engaged by the gaming interface. Engagement of the patient increases the likelihood of the patient performing the VTE prevention exercises, which increases the long-term health of the patient and reduces the likelihood of side effects and reduces unnecessary additional hospital stays.

Other measurements from the wearable sensor may be used to categorize patients as low responders or high responders. For example, a difference in ankle plantar flexion velocity patterns is discernible between patients in the HR and LR groups. While the average velocity of moving from one target to another was similar between the two groups, the peak values of the velocity had different patterns between the two groups. HR group members generally use a more consistent but lower ankle velocity to move the cursor from one target to another target, which results in pumping the blood for a longer period. LR group members generally have a peak ankle velocity raised to a higher level than the HR group, however this is only for a short period of time. FIG. 7 shows a simple diagram representing the differences in these two patterns. A line 702 illustrates ankle velocity changes for high responders; a line 704 illustrates ankle velocity changes for low responders. A median of power spectrum of EMG for MG also mirrored these patterns. The HR members had a lower median frequency when compared to LR groups, thus showing the higher use of slow-twitch fibers in the HR group. Measurements of ankle velocity changes and/or power spectrum of EMG for MG or other muscles may thus be used to categorize patients and identify patients needing further attention.

These game-based exercises illustrate that adding a gaming element to low-cost ankle exercises may help with improvement in blood flow and enhance the patient's compliance by preventing physical and psychological fatigue. The exercise platform also enables feedback for the patient and the caregiver and provides them with progress reports. Progress reports may include information collected by sensors during exercise programs and processed to calculate flow amounts and velocities, information regarding ankle movements collected from motion sensors in the wearable sensor, number of exercise procedures started, number of exercise procedures completed, number of exercise procedures aborted, frequency of completion of exercise procedures, and/or whether exercise procedures have been completed according to a set schedule. Furthermore, the biomechanics of ankle movement have an impact of how blood flow changes to revolutionize the care and prevention for DVT patients.

The schematic flow chart diagram of FIG. 3 is generally set forth as a logical flow chart diagram. As such, the depicted order and labeled steps are indicative of aspects of the disclosed method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagram, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The operations described above as performed by a controller may be performed by any circuit configured to perform the described operations. Such a circuit may be an integrated circuit (IC) constructed on a semiconductor substrate and include logic circuitry, such as transistors configured as logic gates, and memory circuitry, such as transistors and capacitors configured as dynamic random access memory (DRAM), electronically programmable read-only memory (EPROM), or other memory devices. The logic circuitry may be configured through hard-wire connections or through programming by instructions contained in firmware. Further, the logic circuity may be configured as a general purpose processor capable of executing instructions contained in software. If implemented in firmware and/or software, functions described above may be stored as one or more instructions or code on a computer-readable medium. Examples include non-transitory computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise random access memory (RAM), read-only memory (ROM), electrically-erasable programmable read-only memory (EEPROM), compact disc read-only memory (CD-ROM) or other optical disk storage, magnetic disk storage or other magnetic storage devices, or any other medium that can be used to store desired program code in the form of instructions or data structures and that can be accessed by a computer. Disk and disc includes compact discs (CD), laser discs, optical discs, digital versatile discs (DVD), floppy disks and Blu-ray discs. Generally, disks reproduce data magnetically, and discs reproduce data optically. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

Although the present disclosure and certain representative advantages have been described in detail, it should be understood that various changes, substitutions and alterations can be made herein without departing from the spirit and scope of the disclosure as defined by the appended claims. For example, although motor-cognitive impairment testing is described for the iTMT platform, the platform may also be used for motor-cognitive exercise training, assessing risk of falling, predicting outcomes post-intervention, screening outcomes, predicting adverse events such as delirium, studying the brain, and/or evaluating dual tasking on certain brain region activation. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method, comprising:
presenting a series of requested motions to be performed by a patient;
receiving motion data from a wearable sensor recording the patient performing the series of requested motions;
analyzing the received motion data to determine accurate execution of the series of requested motions;
providing feedback to the patient to guide the patient to accurately execute the series of requested motions;
analyzing the received motion data to determine compliance with venous thromboembolism (VTE) preventive measures; and
reporting the determined compliance for the patient.

2. The method of claim 1, wherein the step of receiving motion data from the wearable sensor comprises receiving motion data for a lower extremity joint.

3. The method of claim 2, wherein the step of presenting the series of requested motions comprises presenting a series of targets on a display instructing the patient to move a virtual object on the display through the series of requested motions by moving the lower extremity joint.

4. The method of claim 2, further comprising adjusting at least one of a speed, frequency, and timing of requested motions to the patient based, at least in part, on analyzed patient-specific data.

5. The method of claim 2, further comprising providing real-time feedback in a form of visual, audio, textual, or vibratory feedback is provided during movements to assist the user to optimally execute movements.

6. The method of claim 2, further comprising providing a real-time feedback in a form of visual, audio, textual, or vibratory feedback at an end of a movement execution to inform the user about how well the movement was executed.

7. The method of claim 2, further comprising providing a notification to notify the patient of a next exercise session according to performance and duration of a previous exercise session, clinician recommendation, patient's risk, or patient demographical information.

8. The method of claim 3, further comprising:
receiving venous flow data corresponding to the received motion data; and
analyzing the received venous flow data to determine a risk for deep vein thrombosis (DVT).

9. The method of claim 8, wherein analyzing the received venous flow data comprises determining at least one of flow volume, mean velocity, peak velocity, and vein area.

10. The method of claim 1, wherein the step of analyzing the received motion data comprises determining a total time used by the patient to complete the series of requested motions.

11. A computer program product, comprising:
a non-transitory computer readable medium comprising code for performing steps comprising:
presenting a series of requested motions to be performed by a patient;
receiving motion data from a wearable sensor recording the patient performing the series of requested motions;
analyzing the received motion data to determine venous thromboembolism (VTE) compliance;and
reporting the determined compliance for the patient.

12. The computer program product of claim 11, wherein the step of receiving motion data from the wearable sensor comprises receiving motion data for a lower extremity joint.

13. The computer program product of claim 12, wherein the step of presenting the series of requested motions comprises presenting a series of targets on a display instructing the patient to move a cursor on the display through the series of requested motions by moving the lower extremity joint.

14. The computer program product of claim 13, wherein the medium further comprises code for performing steps comprising:
receiving venous flow data corresponding to the received motion data; and
analyzing the received venous flow data to determine a risk for deep vein thrombosis (DVT).

15. The computer program product of claim 14, wherein analyzing the received venous flow data comprises determining at least one of flow volume, mean velocity, peak velocity, and vein area.

16. The computer program product of claim 11, wherein the step of analyzing the received motion data comprises determining a total time used by the patient to complete the series of requested motions.

17. A system, comprising:
a wearable sensor comprising at least one motion sensor, wherein the wearable sensor is configured to record motion data from the at least one motion sensor and to transmit the motion data; and
a computing device configured to receive the motion data transmitted by the wearable sensor and configured to perform steps comprising:
presenting a series of requested motions to be performed by a patient;
receiving motion data from a wearable sensor recording the patient performing the series of requested motions;
analyzing the received motion data to determine venous thromboembolism (VTE) compliance; and
reporting the determined compliance for the patient.

18. The system of claim 17, wherein the step of receiving motion data from the wearable sensor comprises receiving motion data for a lower extremity joint, wherein the step of analyzing the received motion data comprises determining a total time used by the patient to complete the series of requested motions.

19. The system of claim 18, wherein the step of presenting the series of requested motions comprises presenting a series of targets on a display instructing the patient to move a cursor on the display through the series of requested motions by moving the lower extremity joint.

20. The system of claim 19, wherein the computing device is further configured to perform steps comprising:
receiving venous flow data corresponding to the received motion data; and
analyzing the received venous flow data to determine a risk for deep vein thrombosis (DVT), wherein the received venous flow data comprises determining at least one of flow volume, mean velocity, peak velocity, and vein area.
